(19) United States
(12) Patent Application Publication
(10) Pub. No.: US 2010/0329924 A1
(43) Pub. Date: Dec. 30, 2010

(54) Sponge Sterilizer

(76) Inventor: Debra J. Fogel Harris, Boca Raton, FL (US)

Correspondence Address: JOHN C. SMITH, P.A., 4125 NW 58th LANE, BOCA RATON, FL, 33496, US

(21) Appl. No.: 12/880,024

(22) Filed: Sep. 10, 2010

Related U.S. Application Data
(63) Continuation-in-part of application No. 11/602,740, filed on Nov. 21, 2006, now abandoned.
Application No. 11/602,740 is a continuation of application No. 11/129,913, filed on May 16, 2005, now abandoned.

(51) Int. Cl. A61L 2/10; B01J 19/08

(52) U.S. Cl. 422/5; 422/291

(57) ABSTRACT

A device for sterilizing and deodorizing sponges using a combination of ultraviolet light in conjunction with ozone. The device uses a container, comprising a casing and lid, having a UV-transmissive support structure disposed therein, the UV-transmissive support dividing the interior of the container into a sponge chamber and a lamp chamber, the sponge chamber dimensioned to receive a sponge, a source of ultraviolet light disposed within the lamp chamber, and wherein at least one interior surface of the sponge chamber is reflective to UV light. An ozone lamp is also provided which produces ozone that a) kills any mold on the sponge, and b) eliminates odor caused by contaminants on the sponge

### CROSS REFERENCE TO RELATED APPLICATIONS

This continuation-in-part application is related to, and claims the benefit of, the patent application entitled “Sponge Sterilizer”, filed May 16, 2005, bearing U.S. Ser. No. 11/129,913 and naming Debra Fogel Harris, the named inventor herein, as sole inventor, the contents of which is specifically incorporated by reference herein in its entirety, and patent application entitled “Sponge Sterilizer”, filed Nov. 21, 2006, bearing U.S. Ser. No. 11/602,740 and naming Debra Fogel Harris, the named inventor herein, as sole inventor, the contents of which is specifically incorporated by reference herein in its entirety.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### REFERENCE TO A “SEQUENCE LISTING,” A TABLE, OR A COMPUTER PROGRAM LISTING APPENDIX

Not Applicable.

### BACKGROUND OF THE INVENTION

1. Field of the Disclosure

This disclosure relates to a multimodal sponge sterilization system. In particular, it provides a system that uses UV radiation for bacterial sterilization of sponges in combination with a source of ozone that simultaneously eliminates odor and kills mold.

2. Description of the Related Art

It is well known in the art of bacterial sterilization to use ultraviolet (UV) light to sterilize objects. For example, U.S. Pat. No. 4,906,551 to Beasley et al. discloses a device for sterilizing and storing toothbrushes that includes a container for holding the toothbrushes that uses an ultraviolet ray lamp to apply germicidal radiation to bristles of toothbrushes stored therein.

U.S. Pat. No. 6,811,748 to Ettlinger et al. discloses an object sterilization system that uses a chamber for storing objects that includes a plurality of sources of ultraviolet light emission arranged in the chamber that sterilize the objects placed in the chamber.

A drawback of the prior art is that the units are either not suited to the sterilization of household sponges or are simply too costly to manufacture for household use. Likewise, another problem associated with the prior art is that while ultraviolet light is effective at killing bacteria, it does not address other Issues such as odor and mold. What is needed is a relatively inexpensively manufactured device for sterilizing household sponges by eliminating both bacteria and mold, and which further leaves the sponge with a clean fresh smell.

### BRIEF SUMMARY OF THE INVENTION

A first embodiment of the invention provides a device for sterilizing and deodorizing sponges using a combination of ultraviolet light in conjunction with ozone. The device uses a container, comprising a casing and lid, having a UV-transmissive support structure disposed therein, the UV-transmissive support dividing the interior of the container into a sponge chamber and a lamp chamber, the sponge chamber dimensioned to receive a sponge, a source of ultraviolet light disposed within the lamp chamber, and wherein at least one interior Surface of the sponge chamber is reflective to UV light. An ozone lamp is also provided which produces ozone that a) kills any mold on the sponge, and b) eliminates odor caused by contaminants on the sponge.

In an embodiment of the invention, an optional glass window is provided to allow the user to see that the device is operational while blocking the user's exposure to short wave UV radiation.

### BRIEF DESCRIPTION OF THE SEVERAL VIEWS OF THE DRAWINGS

FIG. 1 is a front cross-sectional view of the invention.

FIG. 2 is a side cross-sectional view of a first embodiment of the invention.

FIG. 3 is a side cross-sectional view of a second embodiment of the invention.

FIG. 4 is a front cross-sectional view of another embodiment of the invention.

FIG. 5 is a cross-sectional view of a top lamp embodiment of the invention having a removable water tray.

FIG. 6 is a top view of a tandem embodiment of the invention.

FIG. 7 is a cross sectional view of another embodiment of the invention.

FIG. 8 shows an embodiment of the supports of FIG. 7.

FIG. 9 shows an alternative preferred embodiment that uses an ozone lamp in combination with a UV lamp to provide a multimodal approach to sterilization of sponges.

FIG. 10 shows an alternative preferred embodiment that uses multiple ozone lamps in combination with multiple UV lamps to provide a multimodal approach to sterilization of sponges.

### DETAILED DESCRIPTION OF THE INVENTION

Disclosed is an apparatus for sterilizing items that are typically highly germ-laden due to their repeated exposure to moisture and dirt. For example, household sponges typically remain moist after use, providing an excellent breeding ground for bacteria. For ease of discussion, this disclosure has focused on the use of household kitchen sponges to illustrate the benefits of the invention. However, those skilled in the art will recognize that this invention can be applicable to a wide range of applications. For example, in a commercial environment such as a restaurant, sponges and washrags are often used to clean tabletops, countertops, and work areas. Their use in this environment presents a substantial risk of spreading terms and disease from one table to another. In fact, in the process of cleaning a table in a restaurant, a contaminated wash rag may actually deliver contaminants to a table which was clean. The advantage provided by the invention is that it would allow a waitress or busboy in a commercial restaurant to sterilize sponges and washrags used to clean tables such that their use did not contribute to spreading germs and other contaminants. In supermarkets, there are a number of departments that require continuous cleaning, such as the deli section, or seafood section. Typically, supermarkets require machinery to be cleaned on a scheduled basis, but it is possible that the cleaning will be done with a sponge or washrag that is contaminated. An advantage provided by the invention is that allows workers in a commercial environment to conveniently ensure that sponges and washrags are free of contaminants. The technology can be used in any area in which contamination is a problem. For example, parents who use cloth diapers can even use this technology after a diaper is washed to ensure that there are no contaminants. As a result, it is the intent of this application to teach a technology which can be used for a wide range of applications where infectious agents are a problem, but teach it in a straightforward manner by discussing its application to a household kitchen environment.

In addition to contaminants, odor is also a problem. A variety of substances for example, food, garlic, onions, etc. can cause unpleasant odor. This invention eliminates this as a problem through the use of ozone that not only eliminates the odor, but also kills mold that might be on an item.

Those skilled in the art will recognize that the ozone lamps and the ultraviolet lamps used for this invention are well known in the art, and can be obtained from a wide variety of commercial sources. As a result, the structure of these lamps will not be discussed in detail here.

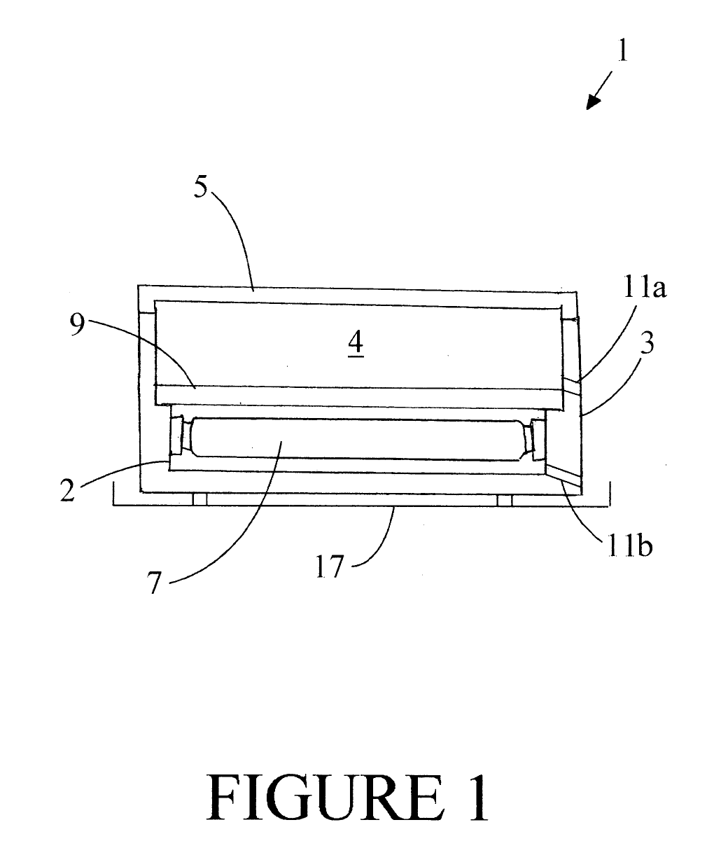

Referring to FIG. 1, there is shown a cross-sectional view of an embodiment of the sponge sterilizer 1 of the invention. A sponge chamber 4 and a lamp chamber 2 are defined by a casing 3 and lid 5, which are sized and dimensioned to hold a short-wave UV lamp 7 in the lamp chamber and a sponge (not shown) in the sponge chamber 4. The two chambers are separated by a UV-transmissive support 9 for supporting the sponge over the UV lamp 7. The sponge chamber 4 is dimensioned for typical household sponges and may be about 1 to 2 inches deep, 4 to 7 inches long, and about 4 to 6 inches wide. Of course, those skilled in the art will recognize that these dimensions may be vary to suit a variety of design objectives. For example, sponge chambers 4 for industrial size sponges will be larger.

The UV-transmissive support 9 may be a solid sheet of a UV-transmissive material, such as quartz, or a simple grid-like structure. If the UV transmissive support is chosen to be solid, then an upper drain hole 11a may be provided to drain away any fluids exuded from the sponge. If the UV-transmissive support is a grid-like structure, then one or more lower drain holes 11b may be provided for the same purpose. Generally, a grid-like structure may be preferred to reduce costs of manufacture. The drain holes 11 will preferable slope downward so as to substantially eliminate the possibility of exposure of a user to the UV radiation emitted by the UV lamp. A small collection tray 17 may be provided to collect drained fluids.

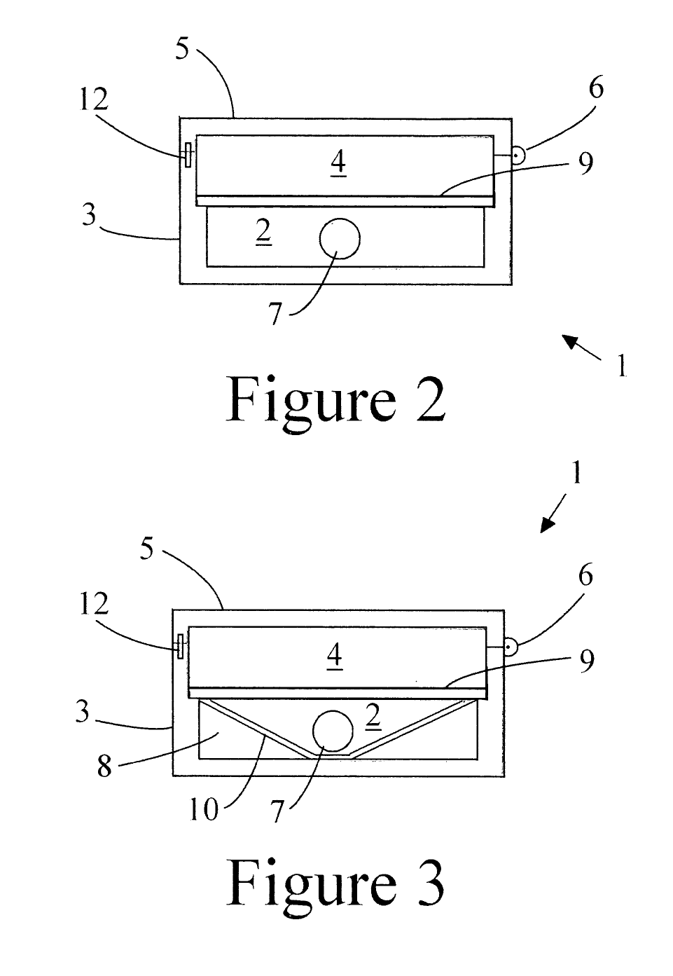

Referring to FIG. 2, there is shown a cross-sectional side view of a first embodiment of the sponge sterilizer I of the invention. Here, the lid 5 is connected to the housing 3 by hinges 6, but the lid may also be designed such that it can be simply be lifted off entirely, thereby further reducing the cost of manufacture. It is preferable that an interlock 12 be provided so as to shut off the UV lamp 7 when the lid 5 is opened. This will prevent exposure of the user to short wave UV radiation. The radiation emitted by the UV lamp may be of wavelengths anywhere from about 200 to about 300 nanometers, generally about 250 nanometers. It is undesirable to expose the human eyes and skin to such wavelengths.

UV lamps are generally expensive, because they must be made of a UV-transparent material, usually quartz. It is desirable, therefore to provide the interior of the sponge chamber 4 with at least one UV-reflective surface so that valuable UV radiation is not wasted by being absorbed into the container walls. Preferably, the lamp chamber 2 will also have at least one reflective surface for the same reason. Further, all of the interior surfaces will preferably be reflective of UV radiation with the exception, of course, of the UV-transmissive support 9. By reflecting the UV radiating within the box, the number of UV lamps 7 needed is reduced. Since the UV lamps 7 are the most expensive single component in the system, this further reduces costs. Further, the UV radiation is essentially trapped in a “light box,” thereby surrounding and “bathing” the sponge in UV radiation and improving the effectiveness of the radiation's sterilizing effect.

One simple way of providing reflective interior surfaces is to simply manufacture the casing 3 and lid 5 of a reflective material such as a metal. For household use, with such use requiring repeated exposure to water, stainless steel may be a suitable choice.

An alternative to stainless steel would be to manufacture the casing 3 and lid 5 of a polymer or glass. The Interior surfaces may then be provided with reflective surfaces, such as by inserting metallic elements, such as plates or foils, or by coating the interiors with a UV reflective material such as by vapor deposition. Suitable materials for reflecting short wave UV are known in the art of vapor deposition, such as aluminum and its alloys, for example aluminum magnesium fluoride (AIMgF₂), silver anodized aluminum, and other coatings known in the UV reflective art. Such coatings may be vapor deposited. Wood and ceramics are also suitable materials for the casing 3 and lid 5. Further, the casing and lid may be manufactured of a polymer that is transparent in the visible spectrum, but blocks UV light. In this case, a window may be formed by leaving a portion of the interior surface uncoated. Such polymers are known in the art and are generally formed by including a UV blocking dye in the polymer during polymerization. An example, a commercially available UV-blocking polymer is sold by the General Electric Company under the trade name LEXAN-SLX.

Alternatively, the UV reflective surface can be curved aluminum with a brushed mirrored aluminum surface to maximize the reflection of UV rays. Preferably, a high-grade aluminum such as commercially available ALZAK™ can be used for the reflective surface. An advantage of using highly reflective material, such as ALZAK, is that it eliminates the need for components such as mirrors.

Referring to FIG. 3, there is shown another embodiment of the invention wherein a reflector element 10 is installed next to the UV-lamp 7 in a manner effective in reflecting UV light emitted by the lamp toward the sponge chamber 4. The reflector element 10 also serves the purpose of creating component chambers 8, wherein the electrical components (not shown) needed to activate the UV-lamp 7 may be disposed. In this manner, the electrical components are protected from any water that might drip from the sponge. Further, the reflector may be shaped and inclined to channel any dripping water to the lower drain hole 11b of FIG. 1. It should be noted that the power supply to the UV lamp 7 may be implemented by batteries, house voltage, or a combination of both (such as when a battery backup is used). Small germicidal UV lamps are known that can generally be powered by as few as four AA batteries.

Note also, that by providing a reflector component 10, the interior surfaces of the lamp chamber 2 need not be reflective, as it is the reflector component that serves this function. This can reduce costs associated with using vapor deposition techniques or expensive materials in the manufacture of the casing 3.

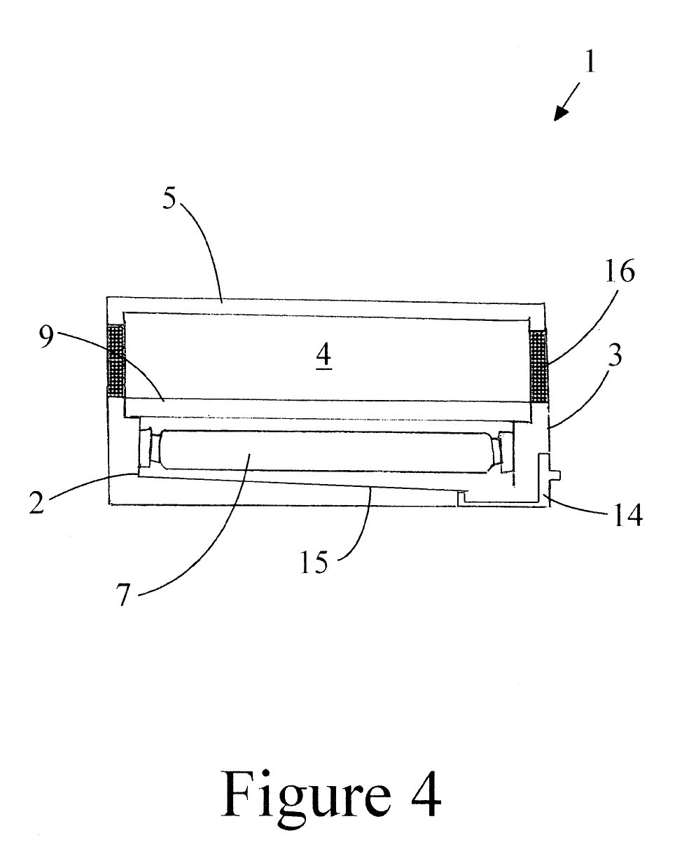

Referring to FIG. 4, there are shown options that may be incorporated with any of the previous embodiments. A sloped low surface 15 in the casing 3 may be provided to direct water to a removable drain tray 14. Additionally, a circumferential glass window 16 may be provided that goes completely or partially around the container. Because it is made of glass, harmful short wave UV radiation is blocked while providing an attractive blue glow that permits the user to see that the device is operational. The circumferential glass window 16 may be vapor deposited with a UV reflective material to improve germicidal effect, yet still transmit light in the visible spectrum.

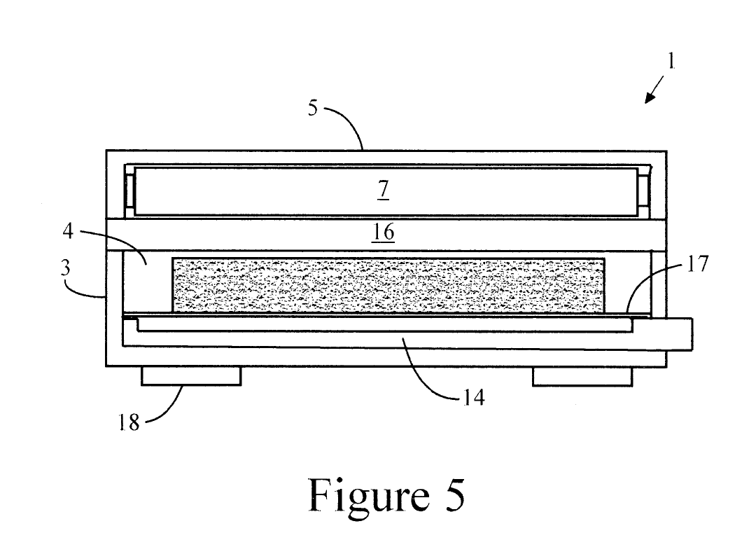

Referring to FIG. 5, there is shown an embodiment with the UV lamp 7 mounted in the lid 5, which eliminates the drainage problems associated with mounting the lamp 7 in the casing 3. The placement of the UV lamp 7 also frees up room for a removable drain tray 14 that extends the length of the sponge chamber. Here the sponge would rest on a perforated grid 17 that permits water to drip into the water tray 14. Rubber feet 18 are also shown in this figure, which may be provided with any of the embodiments in this disclosure.

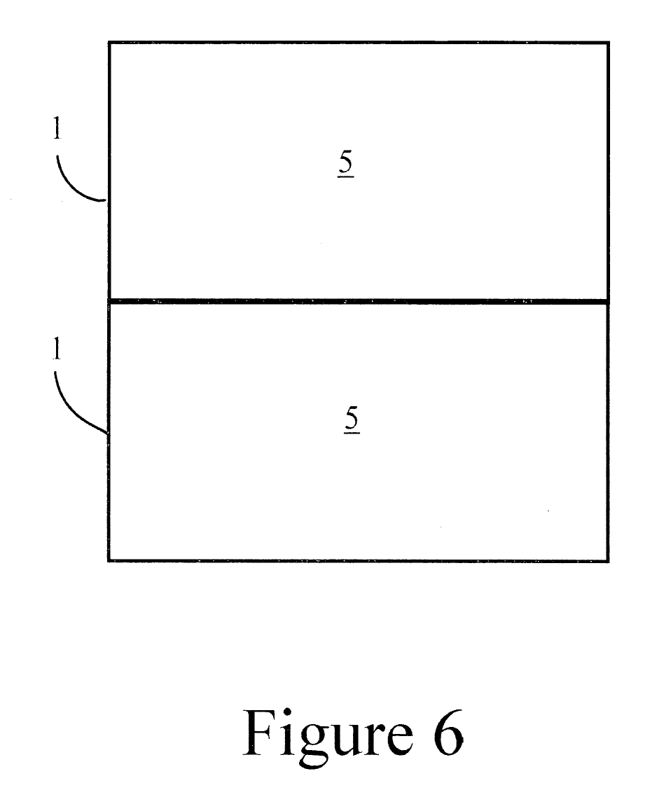

Referring to FIG. 6, there is shown a tandem embodiment of the invention wherein two or more sponge sterilizers 1 are linked together. This may be accomplished by actually affixing together separate sponge sterilizers 1, or molding the entire casing as one piece having multiple sponge chambers. Separate openable lids 5 may be provided for each chamber.

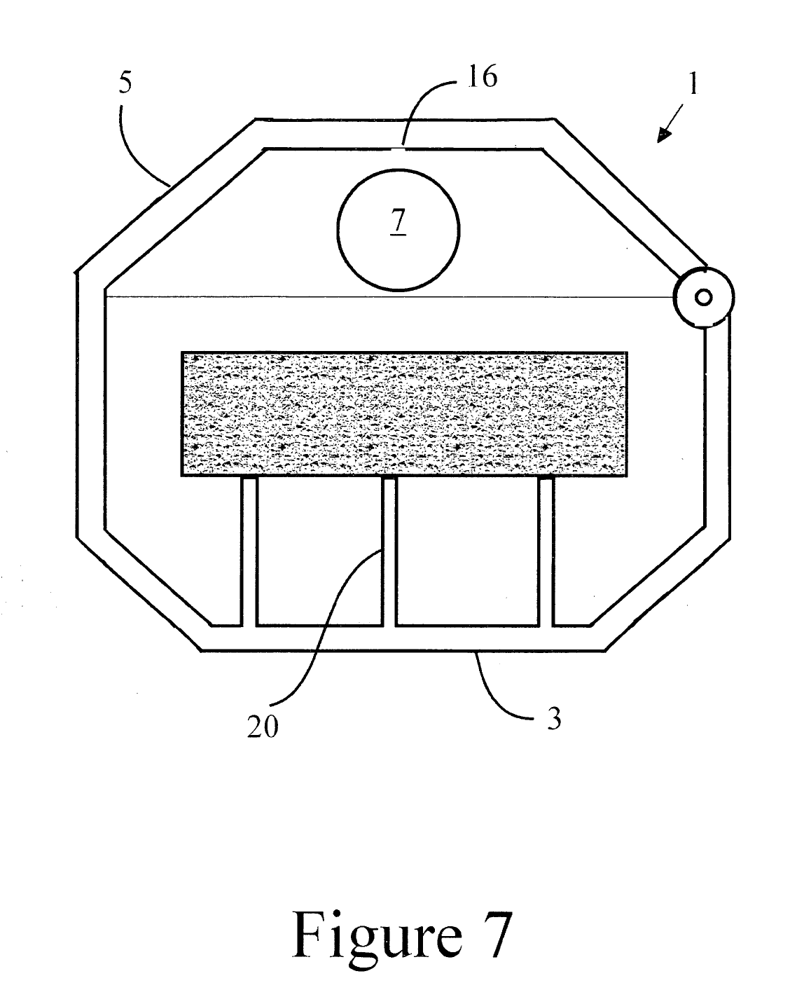

Referring to FIG. 7, there is shown a cut-away end view of an octagonal embodiment of the invention wherein the UV lamp 7 is mounted in the lid and the sponge is supported in the casing by a plurality of supports 20. Because of the angled interior surfaces, UV radiation is effectively reflected around the sides of the interior and underneath the sponge, thereby effectively bathing the sponge in the UV radiation on all sides. Again, the sterilizer may be made of a UV-blocking polymer or glass and a window 16 provided by leaving some portion of the interior surfaces uncoated with UV reflective material.

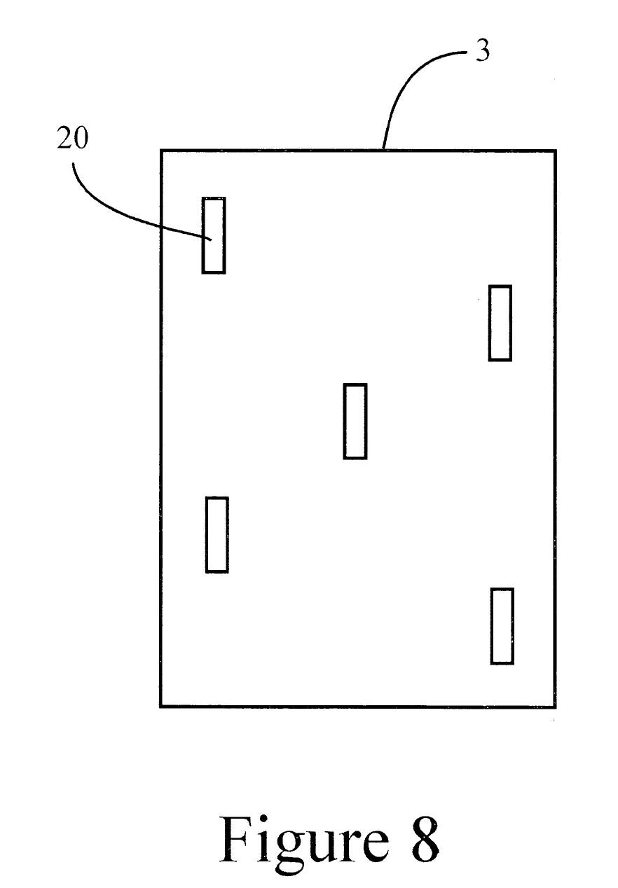

Referring to FIG. 8, there is shown a top view of the supports 20 of FIG. 7. Here, the supports may be optionally staggered to allow UV light to reflect in from the lower angled surfaces without creating “shadow zones” between supports 20, as would be the case if the supports were side-by-side.

As can be seen, a relatively effective sponge sterilizer 1 may be provided at low cost by minimizing the use of short wave UV lamps 7 and providing less costly alternatives to the use of expensive materials.

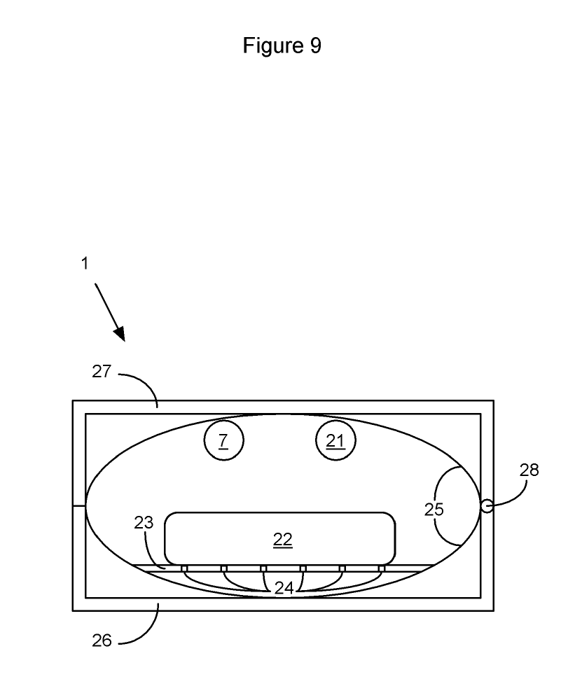

FIG. 9 shows an end view of an alternative preferred embodiment of the sponge sterilizer 1 that uses an ozone lamp 21 in combination with a UV lamp 7 to provide a multimodal approach to sponge sterilization.

In this embodiment, the sponge sterilizer 1 has a lower portion 26 and an upper portion 27. When the sponge sterilizer 1 is opened to insert or withdraw a sponge 22, the upper portion 27 pivots on hinge 28 to allow access to the inner chamber of the sponge sterilizer 1. Alternatively, the hinge 28 can be eliminated and the upper portion 27 can be lifted off of the lower portion 26 as needed.

Preferably, the interior of the sponge sterilizer 1 is fabricated with a highly reflective surface 25, such as ALZAK. Those skilled in the art will recognize that alternative materials can also be used so long as they provide suitable reflectivity.

For ease of discussion, only a single UV lamp 7, and a single ozone lamp 21 is shown. However, those skilled in the art will recognize that a plurality of UV lamps 7 and ozone lamps 21 can be used to ensure more extensive exposure to UV radiation and more ozone production.

This figure also shows a UV-transmissive support rack 23 that supports sponge 22. The material used to fabricate support rack 23 can be anything suitable for its purpose, so long as it is fabricated from material that will allow UV radiation to pass through to the sponge 22. Also shown in this figure are apertures 24 which serve two purposes. First they allow any fluid in the sponge 22 to drain through to a discharge port (not shown) in the lower portion 26. Second, they allow ozone to reach the bottom of the sponge 22. In one preferred embodiment, there would be UV and ozone lamps 26, 27 in the upper portion 27 and the lower portion 26 to enhance distribution of UV radiation and ozone.

The advantage of a multimodal approach is that it first accomplishes a primary goal of killing bacteria through the use of UV radiation. This is very important with items such as kitchen sponges because they tend to be highly contaminated with bacteria. The second goal of the invention is to achieve other advantages not possible solely through the use of UV radiation. By adding ozone to the sterilization process, two additional advantages are achieved: mold elimination and odor reduction. Mold provides a serious health risk. Ozone is useful because it will kill mold present in the sponge 22. In addition, the ozone provides a second benefit in that it eliminates odor. A problem associated with kitchen sponges is that they tend to acquire a bad odor. The ozone actively eliminates the odor from the sponge.

As a result of using the multimodal approach provided by the invention, multiple benefits are achieved: bacterial and viral contaminants are destroyed, mold is destroyed, and odor is eliminated.

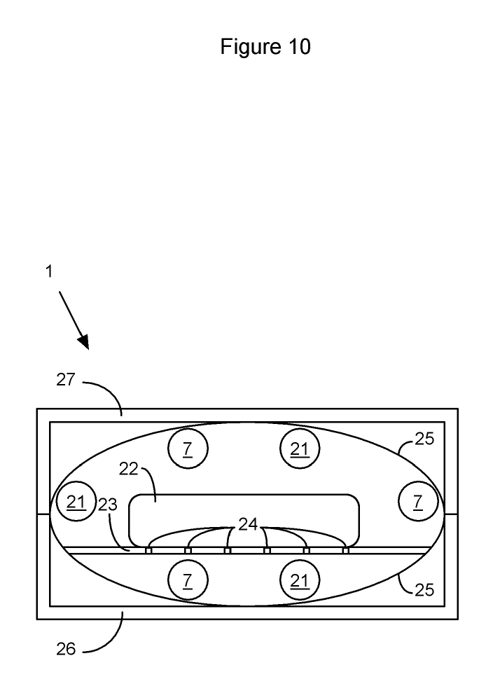

In FIG. 10, a variation of the alternative preferred embodiment of FIG. 9 is shown that uses multiple ozone lamps 21 in combination with multiple UV lamps 7 to provide a more intensive sterilization process. In this embodiment, UV lamps 7 are positioned in multiple locations such that substantially all of the surface area of the sponge 22 is exposed to direct radiation from the UV lamps 7. In addition the reflective surfaces 25 increase the level of UV radiation exposure are reflecting UV radiation back to the sponge 22. Likewise, a plurality of ozone lamps 21 are shown positioned throughout the device to increase the level of ozone concentration.

Those skilled in the art will recognize that multimodal embodiments of FIGS. 9 and 10 can also be implemented with any of the embodiments illustrated in FIGS. 1-8.

While specific embodiments have been discussed to illustrate the invention, it will be understood by those skilled in the art that variations in the embodiments can be made without departing from the spirit of the invention. For example, materials used to fabricate the device can vary, dimensions and shapes of the device can vary based on design choices and applications (i.e., commercial or residential use). Likewise, the configuration of the device may vary due to the nature of the items that a user may want to sterilize, etc. Therefore, the invention shall be limited solely to the scope of the claims. I claim:

### SEQUENCE LISTING

Not Applicable.

1. A multimodal sterilization device for cleaning and deodorizing items, comprising:
a sealable chamber with sufficient size to accommodate an item to be cleaned;
a source of ultraviolet radiation;
a source of ozone; and
a highly reflective surface in the sealable chamber to reflect ultraviolet light inside the sealable chamber;
wherein the ultraviolet radiation destroys bacteria, and the ozone destroys mold and eliminates odor.

2. A device, as in claim 1, further comprising:
a UV-transmissive support rack providing physical support for an item such that the item is held above the bottom of the sealable chamber with sufficient clearance that ultraviolet light can reach substantially all of the surface of the item;
wherein UV radiation can be distributed throughout the sealable chamber from a single UV radiation source.

3. A device, as in claim 2, wherein:
the UV radiation source further comprises at least one UV lamp.

4. A device, as in claim 3, wherein:
the UV radiation source comprises a plurality of UV lamps that are arranged such that UV radiation reaches substantially all of the surface of the item without being reflected.

5. A device, as in claim 4, wherein:
the ozone source comprises at least one ozone lamp.

6. A device, as in claim 5, wherein:
the ozone source comprises a plurality of ozone lamps.

7. A device, as in claim 2, wherein:
the support rack further comprises a one or more apertures to allow fluids in the item to drain from the item.

8. A device, as in claim 1, wherein:
the UV radiation source comprises at least one UV lamp.

9. A device, as in claim 8, wherein:
the UV radiation source comprises a plurality of UV lamps that are arranged such that UV radiation reaches substantially all of the surface of the item without being reflected.

10. A device, as in claim 1, wherein:
the ozone source comprises at least one ozone lamp.

11. A device, as in claim 2, wherein:
the ozone source comprises a plurality of ozone lamps.

12. A device, as in claim 1, wherein:
the highly reflective surface is a mirror.

13. A device, as in claim 1, wherein:
the highly reflective surface is polished aluminum.

14. A device, as in claim 1, wherein:
the highly reflective surface is ALZAK.

15. A method of sterilizing objects, including the steps of:
placing an item to be sterilized in a sealable chamber;
irradiating the item with ultraviolet radiation to kill bacteria; and
exposing the item to ozone to kill mold, and to eliminate odors;
whereby the ultraviolet radiation and the ozone act in concert to kill bacteria, mold and to eliminate odors.

16. A method, as in claim 15, including the additional steps of:
using at least one UV lamp to generate the UV radiation; and
using at least one ozone lamp to generate the ozone.

17. A method, as in claim 16, including the additional steps of:
using a highly reflective surface to reflect UV radiation inside the sealable chamber; and
suspending the item such that substantially all of its surface area is exposed to UV radiation either directly from the UV lamp, or exposed to UV radiation reflected off of the highly reflective surface.

18. A method, as in claim 17, including the additional step of:
using a plurality of UV lamps to directly apply UV radiation to substantially all of the surface area of the item.

19. A method, as in claim 17, including the additional step of:
fabricating the reflective surface from a mirror, or from polished aluminum.

20. A method, as in claim 17, including the additional step of:
fabricating the reflective surface from ALZAK.
